(19) United States
(12) Patent Application Publication
(10) Pub. No.: US 2004/0034375 A1
(43) Pub. Date: Feb. 19, 2004

(54) Fastening system incorporating washer for skull closure

(76) Inventors: Larry R. Ruiz, Lompoc, CA (US); Lawrence L. Hampton, Santa Maria, CA (US); Heather M. Savage, Ventura, CA (US); John Bourne, Ventura, CA (US)

Correspondence Address: SHUMAKER & SIEFFERT, P. A., 8425 SEASONS PARKWAY, SUITE 105, ST. PAUL, MN, 55125, US

(21) Appl. No.: 10/453,897

(22) Filed: Jun. 2, 2003

Related U.S. Application Data
(60) Provisional application No. 60/384,534, filed on May 31, 2002.

(51) Int. Cl. A61B 17/08

(52) U.S. Cl. 606/151

(57) ABSTRACT

The present invention is directed to improvements in fastening systems used to reattach a skull flap removed during brain surgery where such fastening systems include elements, usually flat or disc shaped, placed on both sides of a patient's cranium that are connected by a post, wire, shank or other similar structure. The improvement consists of a washer made of a compliant material that improves the fixation properties of the fastening system elements. The washer in one embodiment is simply a flat disc made of a compliant material with a centrally located hole. In an alternate embodiment, the washer wraps around the flat or disc shaped element of the fastening system. In a further variant of the embodiments described, the washer includes a central protrusion extending away from top side of the washer. In a further alternate embodiment of the washer in all the described embodiments may also be placed on the upper or outer lock element of a fastening system. In a still further embodiment of the invention, the washer takes the form of a cylindrical plug substantially similar to the central protrusion described herein.

This application claims priority from U.S. Provisional Application Serial No. 60/384,534, filed May 31, 2002, the entire content of which is incorporated herein by reference.

### FIELD OF THE INVENTION

The invention relates to a fastening system for reattaching a skull flap after brain surgery.

### BACKGROUND

When most brain surgery is performed, it is necessary to remove a piece of skull to provide access to the brain. This is done with a hand-held, powered surgical tool similar to a small router. After a small hole is made in which the bit of the router is placed, the bit is then guided to cut out the piece of the skull required. The blade cuts a gap of about 1.5 mm to 2 mm wide so that the piece removed does not fit back into its hole exactly. The piece that is removed is referred to as a skull “flap”.

When the brain surgery is completed, this skull flap must be reattached to the skull. There are several methods of doing this. Most commonly, a series of matching small holes are drilled in the edge of the skull and the edge of the flap. Sutures are then passed through the corresponding holes and the flap is secured back into the skull opening from which it was taken. Because the fit is not exact due to the material removed by the router, the flap typically sits slightly below the surface of the skull resulting in a depressed area that is obvious through the skin.

Another common method substitutes stainless steel wire for the suture material and fewer holes are used. There is still the cosmetically objectionable depressed area resulting.

More recently, surgeons have begun to use titanium micro plates and screws that were developed for internal fixation of facial and finger bones. While this method results in a more cosmetic result, it is extremely expensive.

All of these methods generally take thirty minutes to one hour of additional surgery after four to six hours of brain surgery.

There is another, newer method just beginning to be marketed in which a titanium rivet is placed inside the skull with the stem of the rivet passing between the skull and the flap. A large “pop rivet” type tool is used to force a titanium button down over the stem of the rivet, locking the flap and the skull in place between them. Three or four of these rivets and buttons are used to secure the flap in place. This method is faster than any of the other methods and less expensive than the titanium plates, but more expensive than sutures or wire.

A major disadvantage of all of the methods that use metal as a material is that the metal components create large artifacts in computed tomography (CT) scans and plain radiographs that are used for post surgical follow up and diagnosis.

In the early 1970's, a company by the name of Codman & Shurtleff, Inc., of Raynham, Mass., developed and marketed a similar “rivet and button” system made of soft silicone plastic. This was never a commercial success and was soon withdrawn from the market. Silicone is too soft and flexible to provide sure fixation and the buttons on the outside of the skull were so large that they made very unsightly bumps under the skin.

Another approach to attaching a skull flap is disclosed in U.S. Pat. No. 6,022,351 entitled “Skull closure device and procedure” and issued to Paul W. Bremer, Ross L. Bremer, and Scott Gingold on Feb. 8, 2000. In the '351 invention, a fastening system and a method of reattaching a skull flap is disclosed. The fastening system comprises: a fastener element of biocompatible radiolucent rigid plastic material comprising a substantially disc shaped head and a shank, the shank having a plurality of ratchet teeth thereon, and a thickness of about 1-2 mm. The fastening system further comprises a substantially disc shaped lock element of biocompatible radiolucent plastic material having a through extending opening into at least one locking tooth defining at least part of the opening, the at least one locking tooth cooperating with the ratchet teeth to allow the shank to pass through the opening so that the head and lock element can be forced toward each other, but not allowing movement away from each other.

U.S. Pat. No. 6,485,493 to Bremer discloses another skull flap fastening system incorporating ridges to promote more secure fixation of a skull flap between a shank and locking element. U.S. Pat. No. 6,511,482, to Wellisz, describes a cranial bone flap fixation clip. U.S. Pat. No. 6,379,363, to Herrington et al., discloses a cranial clamp mechanism for reattachment of a skull flap. Table 1 below lists documents that disclose devices for skull flap fixation.

TABLE 1

| Patent Number | Inventors | Title |
|---|---|---|
| 6,485,493 | Bremer | Skull Closure |
| 6,511,482 | Wellisz | Cranial Bone Flap Fixation Clip |
| 6,379,363 | Herrington et al. | Method and apparatus for |
| | | reattachment of a cranial flap |
| | | using a cranial clamp |
| 6,022,351 | Bremer et al. | Skull closure device and procedure |

All documents listed in Table 1 above are hereby incorporated by reference herein in their respective entireties. As those of ordinary skill in the art will appreciate readily upon reading the Summary of the Invention, Detailed Description of the Preferred Embodiments and claims set forth below, many of the devices and methods disclosed in the patents of Table 1 may be modified advantageously by using the structures and techniques of the present invention.

### SUMMARY

The present invention is directed to improvements in fastening systems used to reattach a skull flap removed during brain surgery. The invention has certain objects. That is, various embodiments of the present invention provide solutions to one or more problems existing in the prior art with respect to the skull flap fastening systems.

The problems include, for example, instability and trauma associated with existing skull flap fastening systems. For example, skull flap fastening systems that include elements, usually flat or disc shaped, placed on both sides of a patient's cranium that are connected by a post, wire, shank or other similar structure, can be susceptible to lateral movement that undermines the fixation properties of the fastening system. In addition, some existing skull flap fastening systems can cause injury to the dura mater covering the brain.

Various embodiments of the present invention have the object of solving the foregoing problems. For example, it is an object of the present invention to overcome at least some of the disadvantages of the foregoing procedures by providing a fastening system structure that resists lateral movement relative to the skull flap, and thereby enhances the fixation properties of the fastening system. It is a further object of the invention to provide a fastening system that is generally atraumatic, and reduces potential injury to the patient. A fastening system in accordance with the invention promotes traction and stability at an interface between the fastening system and the skull flap to thereby resist lateral movement. Accordingly, as a further object, the invention promotes a more robust interface between the skull flap and fastening system to provide more secure fixation of the skull flap. It is a further object of the invention to provide a fastening system that is generally atraumatic, and reduces potential injury to the patient.

Various embodiments of the invention may possess one or more features capable of fulfilling the above objects. In general, the invention is an improvement in fastening systems used to reattach a skull flap removed during brain surgery where such fastening systems include elements, usually flat or disc shaped, placed on both sides of a patient's cranium that are connected by a post, wire, shank or other similar structure. The improvement consists of a washer made of a compliant material that improves the fixation properties of the fastening system elements. For example, the washer may be effective in resisting lateral migration of the fastening system relative to the skull flap, providing better fixation.

The washer in one embodiment is simply a flat disc made of a compliant material with a centrally located hole. The hole is dimensioned to fit around the post, wire or shank of the fastening system. In use, the washer is slid over the post, wire or shank until the bottom side of the washer comes into contact with the flat or disc shaped head of the fastening system. Then, the flat or disc shaped head with the washer in place, is moved into contact with the patients cranium and secured according to techniques well known for implementing such fastening systems. In an alternate embodiment of the invention, the washer is not disc shaped but instead has a series of lobes used to position the washer and to conform to the gap in the burr hole or kerf between the skull and the skull flap.

In an alternate embodiment, the washer wraps around the flat or disc shaped element of the fastening system. The washer wraps around the flat or disc shaped element by extending around the sides of the flat or disc shaped element via a single or multiple side pieces and a single or multiple lips. In this embodiment as well, a hole extends through the washer and is dimensioned to fit around the post, wire or shank of the fastening system.

In a further variant of the embodiments described, the washer includes a central protrusion extending away from an upper side of the washer. The hole extends through both the flat disc portion of the washer and the central protrusion, and is dimensioned to fit around the post, wire or shank of the fastening system. The central protrusion is intended to extend into the burr bole or the kerf between the skull and the skull flap. Therefore, the central protrusion is preferably dimensioned the same size as or slightly larger than the burr hole or the kerf between the skull and the skull flap.

In a further alternate embodiment of the invention, the washer in all the described embodiments may also be placed on the upper or outer locking element of a fastening system. In a still further embodiment of the invention, the washer takes the form of a cylindrical plug substantially similar to the central protrusion described herein.

In comparison to known implementations of skull flap fastening systems, various embodiments of the present invention may provide one or more advantages. For example, the invention may enhance the fixation properties of the fastening system. The invention may provide improved conformity between a fastening system and a skull, enhanced coefficient of friction, and added protection to the skull and skull flap. In particular, incorporation of a washer promotes conformance to the skull flap and increased surface area contact for added fixation. The washer also may reduce trauma to the patient, e.g., at the dura matter covering the brain. The washer may absorb some of the stresses created by the locking force exerted by the fastening element and the lock element, decoupling the head of the fastening element from the surface of the skull flap. In addition, the invention may achieve such advantages without significantly complicating the fastening system or prolonging the duration of the surgical procedure required to reattach the skull flap. These advantages may result in improved fixation of the skull flap. Increased fixation reduces the lateral migration of the fastening systems, and therefore reduces the likelihood of atraumatic damage to the patient. In addition, the properties of the invention also may help to reduce the time of surgery needed to reattach a skull flap.

It is a primary object of the present invention to provide an improvement to fastening systems and methods used to reattach a skull flap to a skull covering the opening from which it was removed. This and other objects of the invention will become clear from an inspection of the detailed description of the invention and from the appended claims. Throughout the description, like elements are referred to by like reference numbers. An element referred to by a reference number has all the attributes and characteristics of the element as described wherever in the description unless specifically stated otherwise.

The above summary of the present invention is not intended to describe each embodiment or every embodiment of the present invention or each and every feature of the invention. Advantages and attainments, together with a more complete understanding of the invention, will become apparent and appreciated by referring to the following detailed description and claims taken in conjunction with the accompanying drawings.

### BRIEF DESCRIPTION OF DRAWINGS

FIG. 1 is a top plan view of a prior art exemplary fastener element.

FIG. 2 is a front-end view of the fastener element of FIG. 1.

FIG. 3 is a side elevation view of the fastener element of FIGS. 1 and 2.

FIG. 4 is a side view of a prior art lock element with the ratchet teeth of the fastener element illustrated schematically adjacent thereto.

FIG. 5 is a plan view of the lock element of FIG. 4.

FIG. 6 is a side schematic view showing the use of the prior art fastening system of FIGS. 1-5 in a method of reattaching a skull flap.

FIG. 7 is a top plan view of the skull flap reattached with three fasteners according to the prior art fastening system of FIGS. 1 -5.

FIG. 8 is a top view of one embodiment of a washer for use with a fastener element in accordance with the present invention.

FIG. 9 is a side view of the embodiment of a washer for use with a fastener element in accordance with the present invention as shown in FIG. 8.

FIG. 10 is a side elevation view of the prior art fastener system of FIG. 3 with the washer of FIGS. 8 and 9 in place.

FIG. 11 is a side schematic view showing the use of the prior art fastening system of FIGS. 1-5 with the washer of FIGS. 8-9 in a method of reattaching a skull flap.

FIG. 12 is a perspective view of an alternate embodiment of the washer of FIGS. 8-9.

FIG. 13 is a top view of another embodiment of the washer of the present invention.

FIG. 14 is a side view of the embodiment of the washer of the present invention shown in FIG. 13.

FIG. 15 is a perspective view of the embodiment of the washer of the present invention shown in FIGS. 13 and 14.

FIG. 16 is a perspective view of an alternate embodiment of the washer of FIGS. 13-15.

FIG. 17 is a perspective view of the embodiment of the washer of the present invention shown in FIGS. 13-15 in place on the prior art fastener system of FIG. 13.

FIG. 18 is a side elevation view of the prior art fastener system of FIG. 3 with the washer of FIGS. 13-15 in place.

FIG. 19 is a perspective view of the prior art fastener system of FIG. 3 with the of FIGS. 13-15 in place.

FIG. 20 is a perspective view of an alternate embodiment of the washer of FIGS. 13-15.

FIG. 21A is a perspective view of an alternate embodiment of the washer of FIGS. 8-9.

FIG. 21B is a perspective view of an alternate embodiment of the washer of FIGS. 8-9.

FIG. 22 is a perspective view of an alternate embodiment of the washer of FIGS. 13-15.

FIG. 23 is a perspective view of an alternate use of the washer of FIGS. 8-9.

FIG. 24 is a perspective view of an alternate use of the washer of FIGS. 13-15.

FIG. 25 is a perspective view of an alternate embodiment of the washer of the present invention.

FIG. 26 is a perspective view of an alternate embodiment of the washer of FIGS. 13-15.

FIGS. 27A and 27B are perspective views of two embodiments of the washer in FIG. 12.

FIG. 28 is a perspective view of an alternate use of the washer of FIGS. 13-15.

FIG. 29 is a perspective view of a spacer in the context of a fastening system.

FIG. 30 is a perspective view of an alternate spacer in the context of a fastening system.

FIG. 31 is a flow diagram illustrating an exemplary skull closure method in accordance with the invention.

### DETAILED DESCRIPTION OF THE PREFERRED EMBODIMENTS

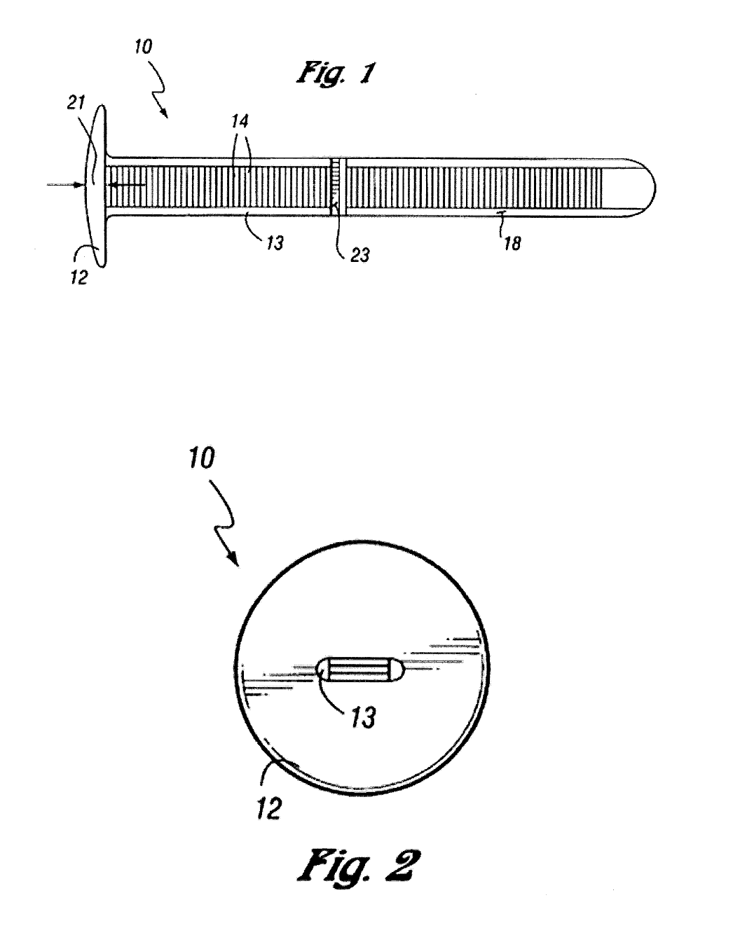

FIGS. 1-5 show a prior art fastening system as described in U.S. Pat. No. 6,022,351, the teachings of which are incorporated herein in their entirety by reference. The fastening system of the '351 patent includes a fastener element 10 and a lock element 11. Both of the elements 10, 11 are made of biocompatible radiolucent rigid plastic material, preferably but not necessarily the same material. An example of suitable material includes acetyl plastic. The material may have a Rockwell hardness of about M94, R120 (ASTM method D785, IS02039) (or.±0.1-4%), and a flex yield strength (ASTM D790) of 99 (or .±1-4%). However, the invention is in no way limited to use with the material described above. While elements 10 and 11 may be made by any suitable technique, they preferably are injection molded.

The fastener element 10 includes a substantially disc shaped head 12, and a shank 13. The term “shank,” as used herein, may refer to any post, wire, shank or other elongated member configured to receive as locking element. The shank 13 has a plurality of ratchet teeth 14 associated therewith. Because of the scale the ratchet teeth 14 are not clearly visible in FIGS. 1 through 3, but are shown schematically in enlarged form in FIG. 4, having a construction that is similar to that of a conventional plastic cable tie, with each ratchet tooth having a ramp surface 15 leading up to a point 16 and then a precipitous drop to a valley 17 on the opposite side of the ramp 15 from the point 16. Preferably, there is at least one set of ratchet teeth 14, but in the preferred embodiment the shank 13 is flat and thin and has ratchet teeth 14 on opposite substantially flat and substantially parallel surfaces thereof. For example, as seen in FIGS. 1 and 3, shank 13 may have a substantially flat top surface 18, and is substantially parallel to a corresponding substantially flat bottom surface 19, with a thickness 20 that is small enough so that the shank 13 may readily fit in the gap between a skull flap and skull from which it has been removed. Typically, the dimension 20 (FIG. 3) of such a gap is between about 1-2 mm.

The ratchet teeth 14 need only be provided on sufficient portions of the surfaces 18, 19 so as to allow effective fastening of the fastener element 10 in place to hold a skull flap in a skull opening. In the preferred embodiment illustrated, however, ratchet teeth 14 are provided over at least approximately the majority of the surfaces 16, 18.

All of the dimensions of the fastener element 10 are specifically designed to allow its effective use in holding a skull flap in place in a skull opening. For example, the head 12 has a small thickness 21 so that it does not have the potential of injuring the dura mater covering the brain. As one example, the dimension 21 may be less than about 3 mm, e.g. about 2.5 mm. The diameter of the substantially disc shaped head 12 can vary fairly widely, as long as it is greater than about 4 mm, but preferably it is between approximately 10 and 20 mm, and more preferably between approximately 13 and 17 mm. The shank 13 may have a length of at least about 3 cm. The dimensions described above are exemplary and should not be considered limiting of the invention as broadly embodied herein.

In the embodiment illustrated in the drawings, the fastener element 13 may have at least one weakened section 23 (e.g. having a thickness significantly less than the thickness 20) at which the shank 13 breaks off, rather than the ratchet teeth breaking, if the fastening system is over-tightened. In some embodiments, the weakened section 23 may be at least about 2 cm from head 12, and shank 13 may have a length of at least about 2 cm past the weakened section 23 (i.e., on the opposite side thereof from the head 12).

The locking element 11 is a substantially disc shaped element having an opening 25 extending therethrough, and the element 11 is also thin. The element 11 should be thin enough so that it does not form an unsightly bump underneath the skin when it is used to hold the skull flap in place in a skull opening from which it has been removed. For example, in some embodiments, the thickness 26 illustrated schematically in FIG. 4 may be less than about 2 mm.

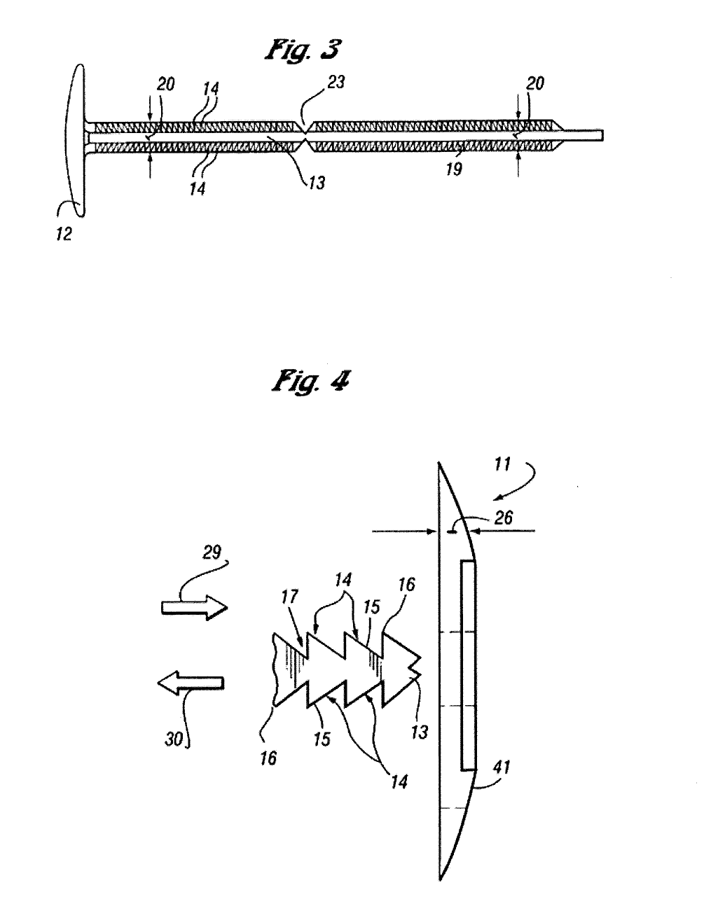

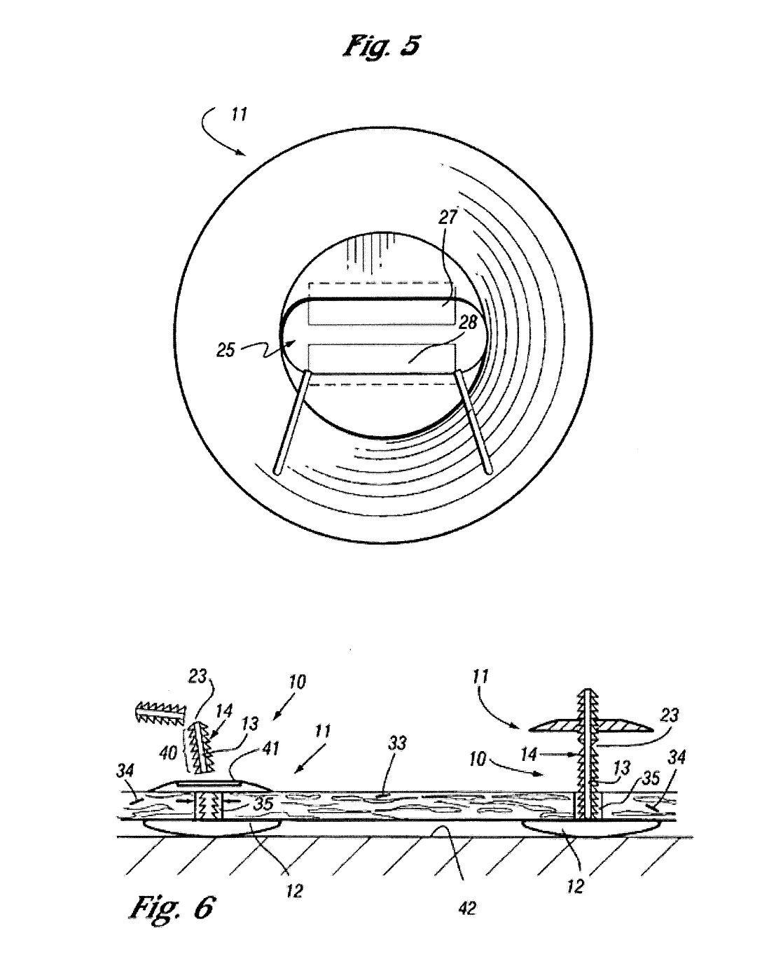

The shank 13 and locking element 11 include a cooperative locking structure for locking engagement. In one embodiment, as described above, the locking structure may include the use of ratcheting teeth. In this example, as shown in FIG. 5, at least part of the opening 25 defines at least one locking tooth 27, and preferably a pair (or more) of locking teeth 27, 28, one locking tooth 27,28 (or set of locking teeth) being associated with each set of ratchet teeth 14 on the surfaces 18, 19. The teeth 27, 28 are designed so that they will flex just enough so that when they are engaged by a ramp face 15 of the ratchet tooth 14, they will move out of the way, but will snap back into place once the point 16 of that tooth 14 has moved therepast. In other words, the locking tooth 27, 28 will fall into the valley 17 between the ratchet tooth 14 and the next tooth 14. However, the teeth 27, 28 will not allow movement in the opposite direction. There may be relative movement between the disc shaped locking element 11 and shank 13 not in the direction 29 illustrated in FIG. 4, but in the direction 30. Once the lock element 11 has been locked into place it can only be removed by destruction thereof. The incorporation of ratchet teeth represents one example of a cooperative locking structure. Alternative structures may incorporate grooves, ridges, threads and the like.

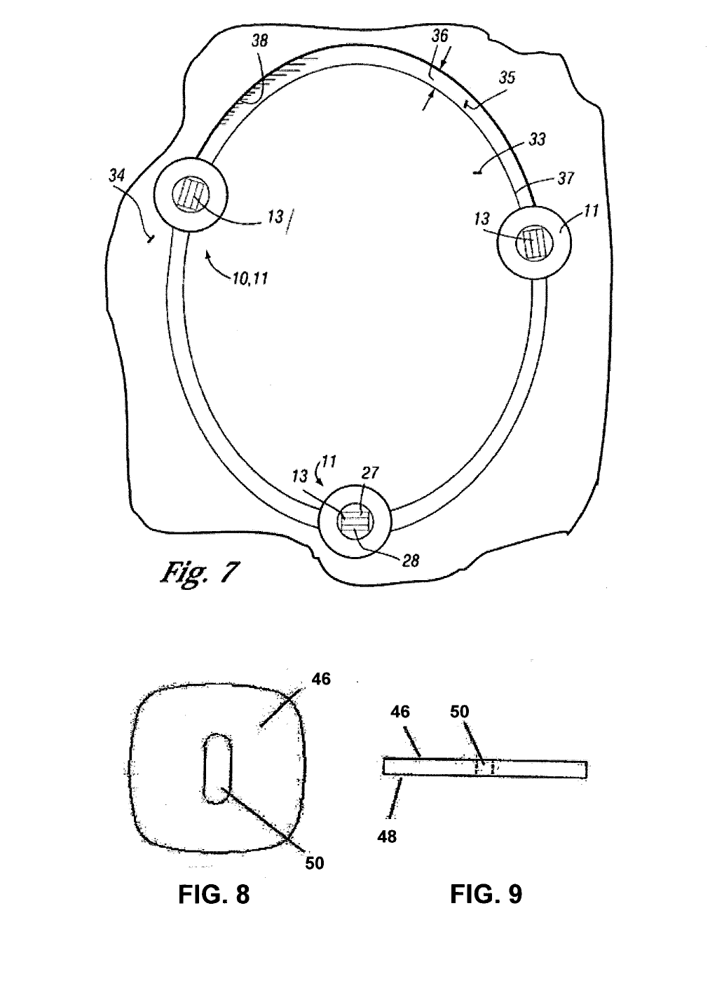

FIG. 6 illustrates schematically the use of the fastening system of FIGS. 1-5 comprising elements 10, 11, to fasten a skull flap 32 into place within an opening in a skull 34 from which it has been removed. As seen in both FIGS. 6 and 7, skull flap 33 is formed by forming gap 35 in the skull 34, which is typically about 1.5-2 mm wide, as indicated by the dimension 36 in FIG. 7. The thickness 20 of the shank 13, at least adjacent the head 12 where the shank 13 fits in the gap 35, is slightly less than the dimension 36. The method is practiced by placing a plurality of the fastener heads 12, with the shanks 13 facing outwardly as illustrated in FIG. 6, in the opening in the skull 34 from which the skull flap 33 as originally removed. The skull flap 33 has the same basic shape as the opening, just slightly smaller in dimension, so as to provide the gap 35. The skull flap 33 is placed in the opening so that the gap 35 is provided between the outer periphery 37 of the skull flap 33, and the periphery 38 of the skull opening. The fastener shanks 13 extend outward through the gap 35.

The lock elements 11 are placed over the shanks 13, and then for each of the fasteners 10 the lock element 11 is forced toward the head 12 so that the ratchet teeth 14 and the locking teeth 27, 28 move with respect to each other until the head 12 and lock element 11 are locked together holding the skull flap 33 in a position closing the skull opening, as seen for the left side elements 10, 11 in FIG. 6, and as shown for all of the elements in FIG. 7. A forcing action may be provided in any suitable way, but preferably is accomplished using a tool that is substantially similar to a conventional pop rivet tool which holds the shank 13 steady while it pushes the lock element 11 toward the head 12. Such a tool may be modified to accommodate the dimensions and the particular shape and construction of locking element 11 and shank 13.

Finally, substantially all shank 13 portions extending outwardly from the lock elements 11 are removed. In the preferred method, utilizing the preferred shanks 13 according to the invention, the tool that is used to force the lock element 11 to and the head 12 will cause the shank 13 to be stressed so that it breaks at the weakened portion 23, as schematically illustrated in the left side of FIG. 6, indicating that the fastener has been properly tightened. The remaining portion of the shank 13 that extends away from the lock element 11, as illustrated at 40 in FIG. 6, is then removed by cutting it off, with a blade, a heating unit, or in any other suitable conventional manner, so that the outer face 41 of the disc shaped lock element 11 is substantially flush. Then the skin/scalp is placed back over the elements 11.

FIG. 7 schematically illustrates three fastener systems, each comprising fastener element 10 and a locking element 11, according to the present invention properly holding the skull flap 33 in place in the skull opening having the interior periphery 38. In the preferred embodiment according to the invention only three or four element sets 10, 11 (three being shown in FIG. 7) are necessary in order to effectively hold the skull flap 33 in place, and preferably the three or four fastening systems defined by the elements 10, 11 are substantially the only structures holding the skull flap properly in place. Also, because of the particular dimensions and construction of the components, the heads 12 do not injure the dura mater 42 (see FIG. 6) and the locking elements 11 do not provide unsightly bumps when the skin is placed over them. Also, because the elements 10, 11 are preferably radiolucent, they do not produce significant artifacts when the patient has a CT scan or a plain radiograph.

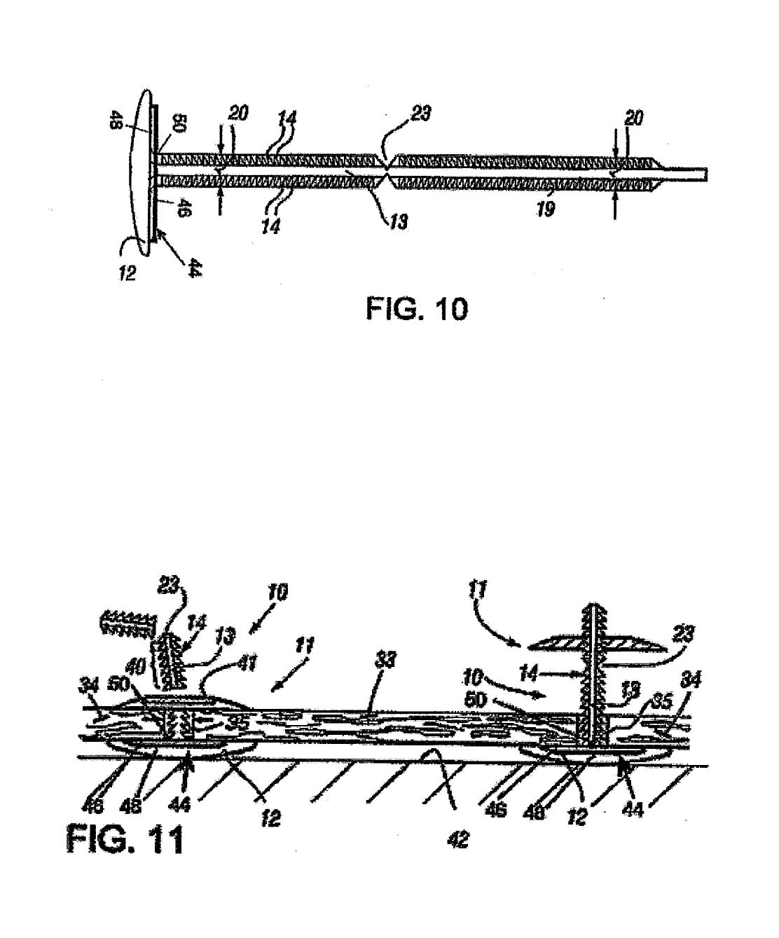

It has been found, however, that it is desirable to improve the general fixation properties of the fastening system. To this end, the present invention provides an improvement to the fastening system described with respect to FIGS. 1-7. This improvement in the general fixation properties of the fastening system is accomplished by adding a washer 44 to the disc shaped head 12 as shown in FIGS. 8 and 9. Washer 44 may be formed from a compliant material such as silicone. Washer 44 may be substantially circular, rectangular, or ovular. In the embodiment of FIGS. 8 and 9, washer 44 is simply a flat disc having topside 46, a bottom side 48 and a hole 50 that accommodates passage of shank 13 of fastener element 10. Washer 44 may have a thickness of about 0.020 inches, but could also be much thicker or thinner. Hole 50 is dimensioned to fit around shank 13. In use, washer 44 is slid over shank 13 until bottom side 48 comes into contact with disc shaped head 12 (FIG. 10). Then, fastener element 11, with washer 44 in place, is moved into contact with the cranium of the patient and secured with locking element 12 as described above and as shown in FIG. 11.

FIG. 12 shows a variant of the washer 44 of FIGS. 8-9. In this embodiment, disc shaped head 12 is not a circular or rectangular disc but instead has a series of lobes 51. Lobes 51 help to position washer 44 and to conform to the gap 35 to which the washer 44 is placed. It is clear that any number of lobes 51 may be used depending on the particular shape of the gap 35 that it is desired to place washer 44.

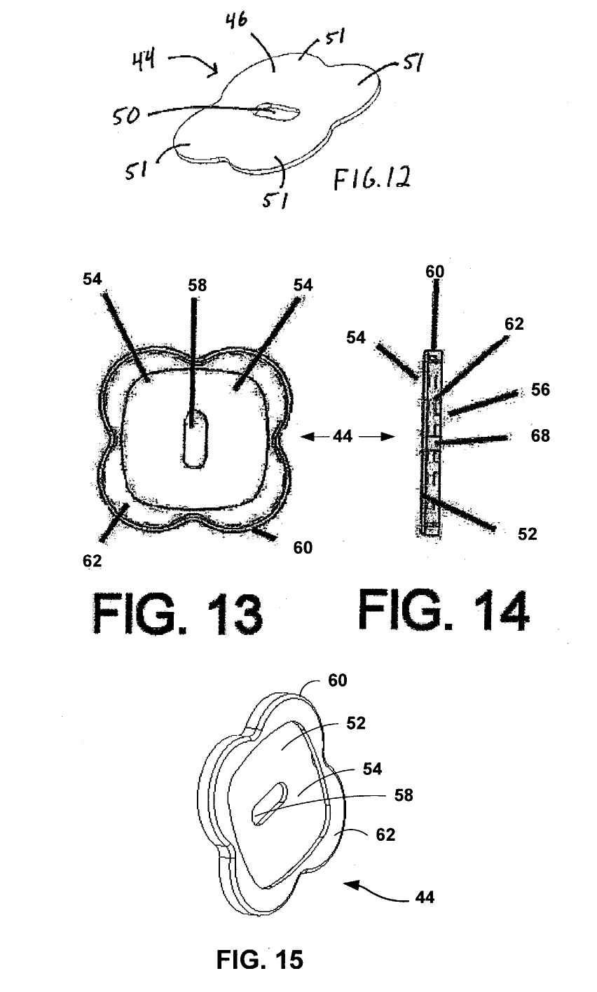

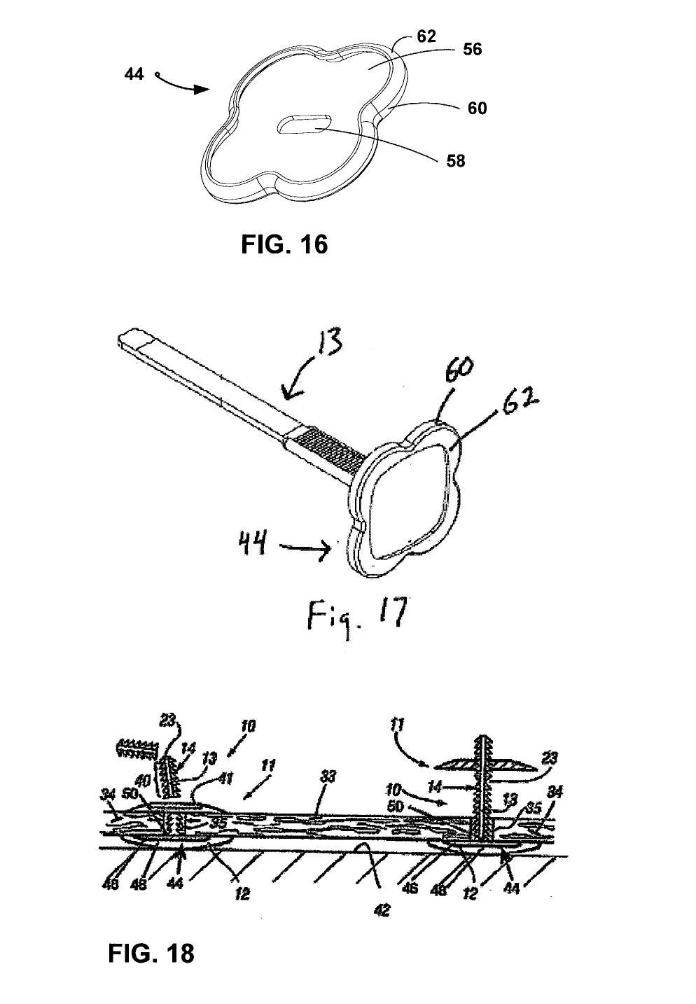

FIGS. 13-15 show washer 44 in an alternate embodiment. In the embodiment of FIGS. 13-15, washer 44 has a substantially flat disc portion 52 having a top side 54, a bottom side 56 and a hole 58. In addition, flat disc portion 52 washer 44 defines, in combination with side piece 60 and lip 62, a recessed area to receive head 12. In particular, washer 44 extends around the sides of disc shaped head 12 via side piece 60 and lip 62. Side piece 60 has about the same thickness as the thickness of disc shaped head 12. Lip 62 extends across the bottom surface of disc shaped head 12 a sufficient distance to allow lip 62 to hold washer 44 in place on disc shaped head. FIG. 16 shows a variant of washer 44 in this embodiment with lip 62 extending only a small distance from side piece 60. It is also clear that lip 62 can extend substantially across the entire bottom surface of disc shaped head 12. Hole 58 is dimensioned to fit around shank 13.

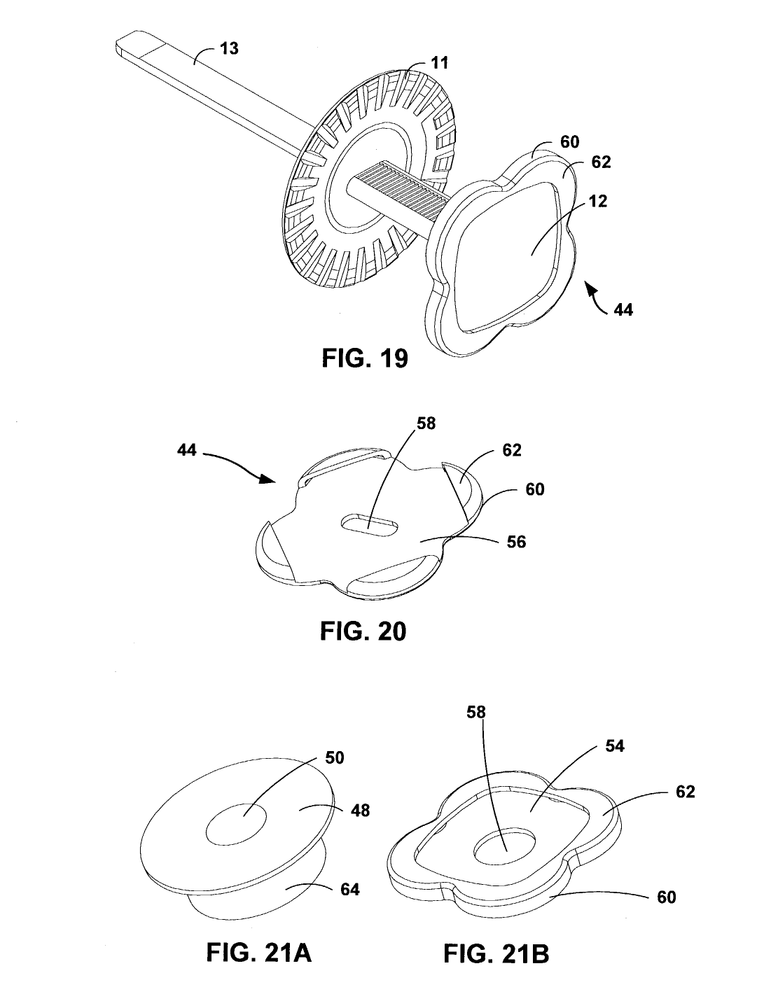

In use, washer 44 is slid over shank 13 until bottom side 56 within the recessed area of flat disc portion 52 comes into contact with disc shaped head 12. Lip 62 is moved over the edge of disc shaped head 12 until lip 62 is moved into contact with the side of disc shaped head 12 opposite the side of disc shaped head 12 that bottom side 56 with which is in contact (FIG. 17). In the configuration, substantially the entire surface of disc shaped head 12 is surrounded by disc portion 52, side piece 60. Then, fastener element 11, with washer 44 in place, is moved into contact with the patient's cranium and secured as described above and as shown in FIGS. 18-19. The shape of lip 62 and the shape of opening 58, to the extent the opening is not circular or square, provides a keying shape that ensures proper alignment between washer 44 and head 12.

FIG. 20 shows an alternate embodiment to the washer of FIGS. 13-15. In this embodiment, portions of side piece 60 and lip 62 are removed. For some uses, this allows the washer 44 to be more easily placed on and wrapped around the disc shaped head 12.

FIG. 21 A shows an alternate embodiment of washer 44. In this embodiment, washer 44 is again substantially a flat disc having topside 46, a bottom side 4 and a hole 50. However, washer 44 also includes a central protrusion 64 by extending away from top side 46. Hole 50 extends through both the flat disc portion of washer 44 and central protrusion 64 and is dimensioned to fit around shank 13. Central protrusion 64 is intended to extend into the gap 35. In all other respects, including use of washer 44 in this embodiment, washer 44 is as described above. In this embodiment, although the external profile of the fastening system is increased, there is an added benefit of the washer 44 extending around the edge of the disc shaped head 12 in that the washer 44 provides protection to the dura mater.

FIG. 21B shows an alternate embodiment of washer 44 shown in FIGS. 13-15. The washer 44 shown in FIG. 21B depicts the hole 58 as circular rather than ovular, as it is in FIG. 15. This is one more exemplary embodiment of the shape of the hole 58 in the washer 44.

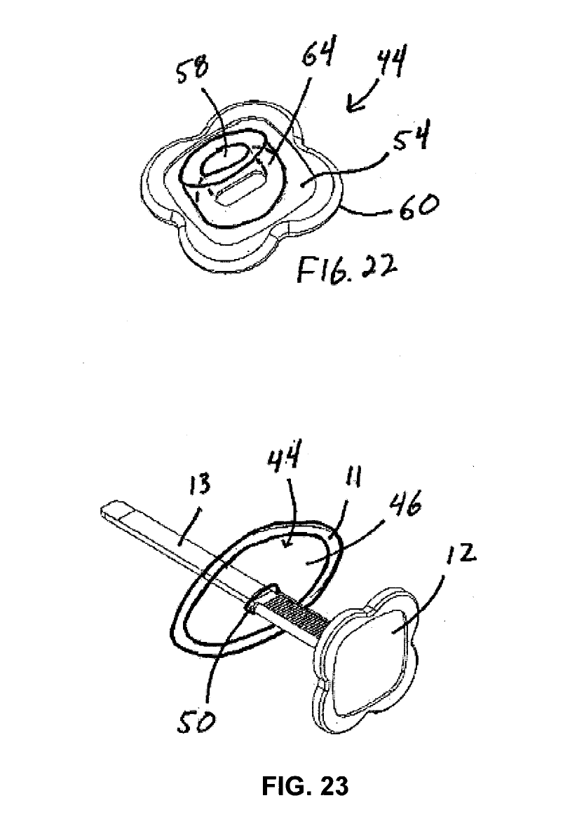

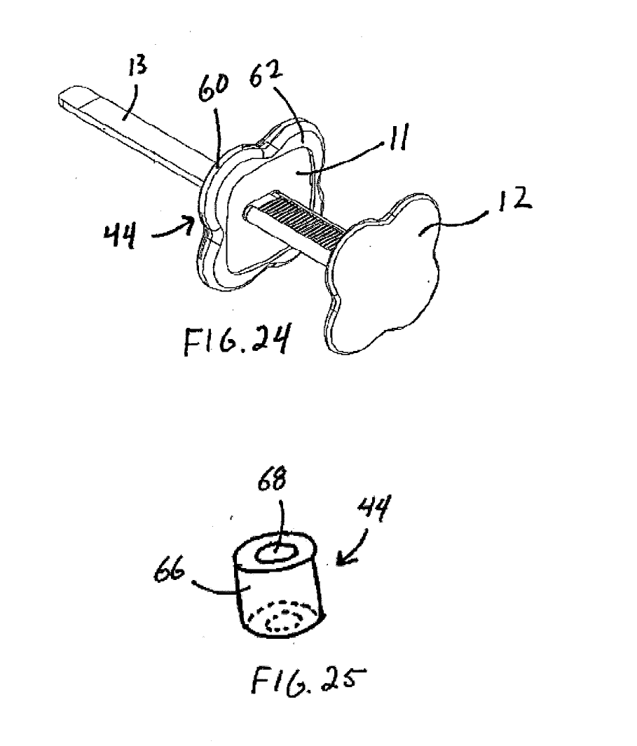

FIG. 22 shows an alternate embodiment of the washer 44 of FIGS. 13-15 wherein the central protrusion 64 is attached to the washer 44 of FIGS. 13-15. In addition to use on disc shaped head 12, washer 44 in all the described embodiments may also be placed on lock element 11 alone or in combination with a washer 44 on lock element 12. FIG. 23 shows the washer 44 of FIGS. 8-9 in place on lock element 11 as well as the washer of FIGS. 13-15 on lock element 12. FIG. 24 shows the washer 44 of FIGS. 13-15 in place on lock element 11.

In a further embodiment of the invention shown in FIG. 25, a cylindrical plug 66 substantially similar to the central protrusion 64 is used as one embodiment of washer 44. In this embodiment, plug 66 is has a central hole 68 that is dimensioned to fit around shank 13. In use, plug 66 is slid over shank 13 until it comes into contact with disc shaped head 12. Then, fastener element 11, with plug 66 in place, is moved into contact with the cranium of the patient and secured as described above.

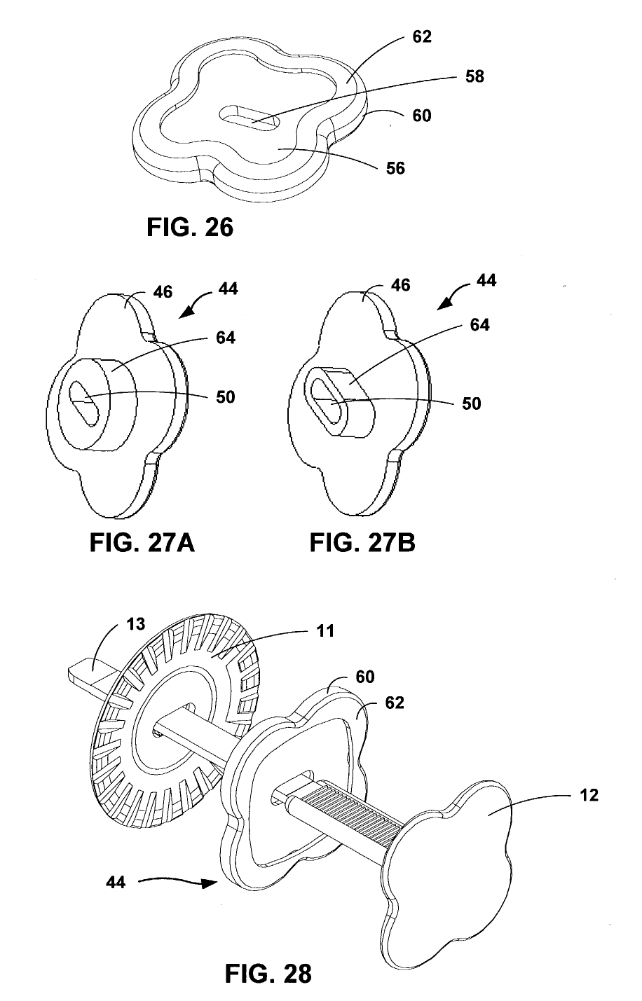

FIG. 26 is a perspective view of an alternate embodiment of the washer of FIGS. 13-15, wherein washer 44 has a substantially flat disc portion 52 having a top side 54, a bottom side 56 and a hole 58. In the embodiment of FIG. 26, top side 54 and bottom side 56 take the form of several lobes rather than a disk. In addition, washer 44 extends around the sides of disc shaped head 12 via side piece 60 and lip 62. Side piece 60 has about the same thickness as the thickness of disc shaped head 12. Lip 62 extends across the bottom surface of disc shaped head 12 a sufficient distance to allow lip 62 to hold washer 44 in place on disc shaped head. It is also clear that lip 62 can extend substantially across the entire bottom surface of disc shaped head 12. Hole 58 is dimensioned to fit around shank 13.

FIGS. 27A and 27B are perspective views of two embodiments of the washer in FIG. 12. FIGS. 27A and 27B shows an alternate embodiment of the washer 44 of FIGS. 12, wherein the central protrusion 64 is attached to the washer 44 of FIGS. 12. In addition to use on disc shaped head 12, washer 44 in all the described embodiments may also be placed on lock element 11 alone or in combination with a washer 44 on lock element 12.

FIG. 28 is a perspective view of an alternate use of the washer of FIGS. 13-15. FIG. 28 illustrates a fastener system including locking element 11, washer 44, fastener shank 13, and fastener head 12.

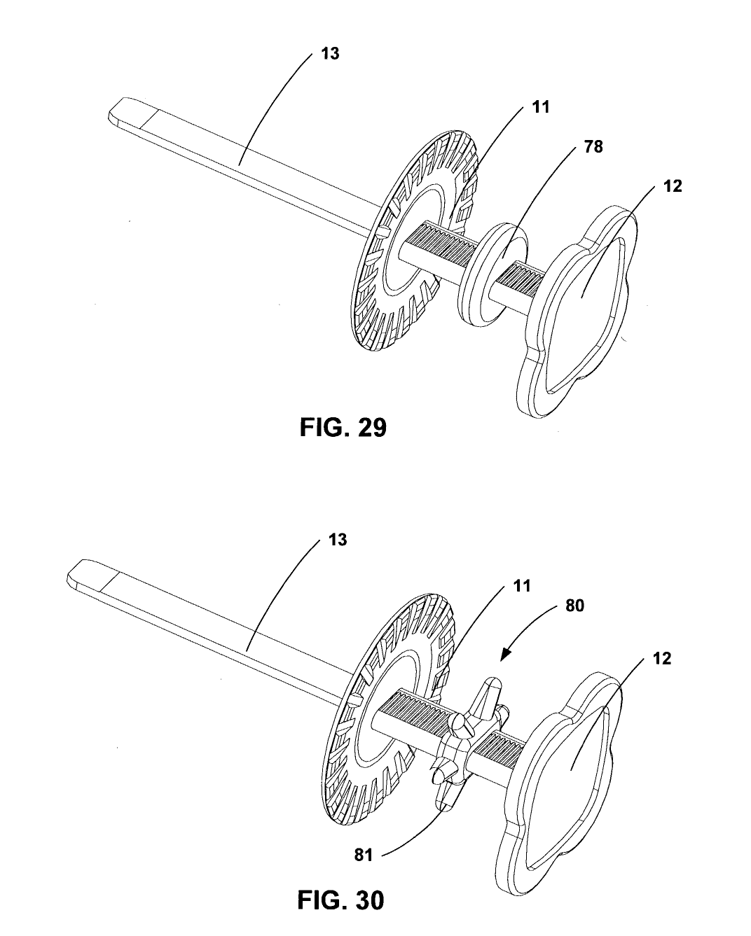

FIG. 29 is a perspective view of a spacer in the context of a fastening system. FIG. 29 illustrates a fastener system including locking element 11, washer 44, fastener shank 13, fastener head 12, and circular spacer 78. The spacer 78 is designed to be used in the gap between the skull and the skull flap in conjunction with a skull flap fastener system. Filling the gap between the skull and skull flap may improve the fixation properties of a fastening system by keeping the fastening system centered in the gap. The spacer 78 may also improve the skull flap reattachment procedure by maintaining the position of the fastener system in the gap and may assist in proper placement of the skull flap. The spacer 78 is used for assembling a fastening system. In one embodiment, the spacer 78 is slipped over the shank and rests between the dura membrane and the skull. After the shank and spacer are cooperating in accordance with the invention, a lock element is slipped over the shank to secure the skull flap in a manner consistent with the principles of the invention.

FIG. 30 is a perspective view of a spacer in the context of a fastening system. FIG. 30 illustrates a fastener system including locking element 11, washer 44, fastener shank 13, fastener head 12, and hub-like spacer 80. Spacer 80 includes a plurality of spoke-like extensions 81 that extend from its center. Spacer 80 behaves much like spacer 78 from FIG. 29. The extensions on spacer 80 may be convenient for certain instances to provide improved fixation.

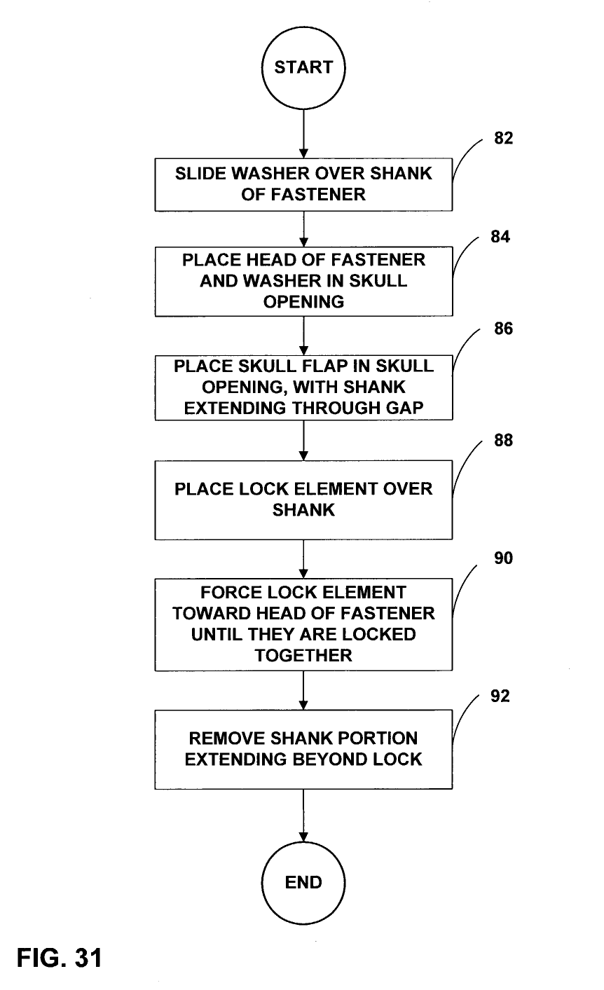

FIG. 31 is a flow diagram illustrating an exemplary skull closure method in accordance with the invention. First, a washer 44 is slid over shank 13 of a fastening element (82). The head of the fastener 12 and the washer 44 are placed in the skull opening (84). After the head 12 and washer 44 are positioned below the skull opening with the shank 13 of the fastener pointing out of the skull, the skull flap is placed in the skull opening with the shank 13 extending through the gap (86). Upon placement of the skull flap in the skull opening, lock element 11 is placed over the shank 13 (88). The lock element 11 is forced down the shank 13 toward the head of the fastener 12 until the lock element and fastener head are locked together (90). The locking of the lock element and the fastener head act to secure the skull flap in the skull opening. After the skull flap is secured, a portion of the shank 13 extends beyond the lock element 11. This portion of the shank is removed (92).

The method depicted in FIG. 31 is merely one example of a method that would use the principles of the invention. Several of the steps in the method may be interchanged, while still conforming to the ideas covered in the invention. For example, the locking element could be placed on the shank before the skull flap is placed in the skull opening.

Washer 44 may be made of a compliant material such as silicone. In particular, a suitable material for washer 44 is a catalyzed silicone elastomer, which is a compliant material. Although the preferred material for washer 44 is silicone, other biocompatible compliant materials such as elastomeric materials may be used as will be clear to those skilled in the art. Because washer 44 is made of a compliant material, washer 44 provides improved conformity to the cranium and cranial flap. Further, washer 44 modifies the effective coefficient of friction of the disc shaped head 12 base material for improved fixation. In addition, the compliant washer 44 allows the disc shaped head 12 to partially squeeze into and conform form within the gap 35 of the burr hole and/or kerf, effectively wedging the cranial flap and fastener system locks into a fixed position.

The current invention, although described above in connection with a specific fastener system is also intended to be applied to any fastener system that has elements on both sides of a patient's cranium that are connected by a post, wire, shank or other similar structure. The current invention not only improves the fixation properties of a fastener system but also improves the seal around the burr hole, kerf or gap 35. In addition, the current invention helps to improve the current surgical techniques available for securing a cranial flap by reducing the total time required in the operating room.

In addition, washers 44 could be used with burr hole caps to secure such burr holes, with cranial or bone plates between such plates and the bone to secure such plates, between screws or other implements used to secure object s or tissue to bone or other rigid or semi-rigid material or tissue and placed between objects, devices, tissue or other objects and the dura or other tissue to prevent traumatic injuries.

The preceding specific embodiments are illustrative of the practice of the invention. It is to be understood, therefore, that other expedients known to those skilled in the art or disclosed herein may be employed without departing from the invention or the scope of the claims. For example, the present invention further includes within its scope methods of making and using systems for transurethral ablation, as described herein.

In the claims, means-plus-function clauses are intended to cover the structures described herein as performing the recited function and not only structural equivalents but also equivalent structures. Thus, although a nail and a screw may not be structural equivalents in that a nail employs a cylindrical surface to secure wooden parts together, whereas a screw employs a helical surface, in the environment of fastening wooden parts a nail and a screw are equivalent structures.

Many embodiments of the invention have been described. Various modifications may be made without departing from the scope of the claims. These and other embodiments are within the scope of the following claims.

1. A skull flap fastening system comprising:
a fastener element of biocompatible material having a head and a shank;
a washer of biocompatible, compliant material having a first opening sized for passage of the shank; and
a lock element of biocompatible material having a second opening sized for passage of the shank and a locking structure for locking engagement, the fastener element and the lock element cooperating to secure a skull flap between the lock element and the washer.

2. The system of claim 1, wherein each of the fastener element, the washer and the lock element is formed from a radiolucent material.

3. The system of claim 1, wherein the head of the fastener element is substantially disc-shaped, and the shank has substantially flat first and second substantially parallel surfaces, the surfaces defining ratchet teeth, the lock element.

4. The system of claim 1, wherein the washer has a substantially compliant structure to allow the washer to at least partially conform to a surface of the skull flap adjacent the washer.

5. The system of claim 1, wherein the washer has a substantially compliant structure to at least partially absorb stress generated by engagement of the skull flap between the lock element and the washer.

6. The system of claim 1, wherein the washer material has a coefficient of friction greater than a coefficient of friction of the fastener element.

7. The system of claim 1, wherein the washer includes a catalyzed silicone elastomer.

8. The system of claim 1, wherein the washer defines a recessed area to receive a portion of the head of the fastener element.

9. The system of claim 1, further comprising a central protrusion that extends from the washer to fit within a gap between a skull and the skull flap.

10. The system of claim 1, wherein the washer is formed from a compliant coating applied to the head of the fastener element.

11. The system of claim 1, wherein the washer is substantially annular and disc-shaped.

12. The system of claim 1, wherein the washer defines a plurality of lobes shaped to enhance conformance of the washer to a gap between a skull and the skull flap.

13. The system of claim 1, wherein the washer includes a recessed region that receives the head of the fastener element, and a peripheral lip that extends about sides of the head.

14. The system of claim 1, wherein the shank defines at least one of a series of ratcheting teeth, threads, and ridges to promote locking engagement with the locking element.

15. The system of claim 1, wherein the head of the fastener element includes acetyl, and the shank of the fastener element include polypropylene.

16. The system of claim 1, wherein the washer has a diameter in a range of approximately 13 mm to approximately 17 mm.

17. The system of claim 1, wherein the washer is integrally formed with the head of the fastener element.

18. The system of claim 1, further comprising a plurality of the fastener elements, a plurality of the washers, and a plurality of the locking elements to fasten the skull cap to a skull at multiple positions around a perimeter of the skull cap.

19. A method for reattaching a skull flap, removed from the skull for the purpose of surgical access to the brain, the skull flap having an area smaller than the area of the opening in the skull, the method comprising:
sliding a washer over a shank of a fastener element until the washer comes into contact with a head of the fastener element;
placing the fastener element and the washer within the skull opening with the shank of the fastener facing outward from the opening;
placing the skull flap in the skull opening so that a gap exists between the outer periphery of the skull flap and the periphery of the skull opening, wherein the shank of the fastener element extends through the gap; and
placing a lock element over the shank to secure the skull flap between the lock element and the washer.

20. The method of claim 19, further comprising removing substantially all of a portion of the shank extending outwardly from the locking elements.

21. The method of claim 19, further comprising placing a plurality of the fastener elements, a plurality of the washers and a plurality of the lock elements to secure the skull flap at multiple positions about a perimeter of the skull flap.

22. The method of claim 19, wherein each of the fastener element, the washer and the lock element is formed from a radiolucent material.

23. The method of claim 19, wherein the head of the fastener element is substantially disc-shaped, and the shank has substantially flat first and second substantially parallel surfaces, the surfaces defining ratchet teeth, the lock element.

24. The method of claim 19, wherein the washer has a substantially compliant structure to allow the washer to at least partially conform to a surface of the skull flap adjacent the washer.

25. The method of claim 19, wherein the washer has a substantially compliant structure to at least partially absorb stress generated by engagement of the skull flap between the lock element and the washer.

26. The method of claim 19, wherein the washer material has a coefficient of friction greater than a coefficient of friction of the fastener element.

27. The method of claim 19, wherein the washer is formed from a compliant coating applied to the head of the fastener element.

28. The method of claim 19, wherein the washer is substantially annular and disc-shaped.

29. The method of claim 19, wherein the washer defines a plurality of lobes shaped to enhance conformance of the washer to a gap between a skull and the skull flap.

30. The method of claim 19, wherein the washer includes a recessed region that receives the head of the fastener element, and a peripheral lip that extends about sides of the head.

31. The method of claim 19, wherein the shank defines at least one of a series of ratcheting teeth, threads, and ridges to promote locking engagement with the locking element.

32. The method of claim 19, wherein the washer has a diameter in a range of approximately 13 mm to approximately 17 mm.

33. A skull flap fastening system comprising:
a fastener element of biocompatible material having a head and a shank;
a lock element of biocompatible material having an opening sized for passage of the shank and a locking structure for locking engagement, the fastener element and the lock element cooperating to secure a skull flap between the lock element and the fastener element;
and
means for distributing locking pressure exerted by the head of the fastener element over a portion of the skull flap.

34. The system of claim 33, wherein the means for distributing locking pressure includes a compliant material that substantially conforms to a surface of the skull flap to promote fixation of the head and the lock element relative to the skull flap.

35. The system of claim 33, wherein the means for distributing locking pressure includes a compliant material that enhances friction at an interface between the head and the skull flap to promote fixation of the head and the lock element relative to the skull flap.

36. The system of claim 33, wherein the means for distributing locking pressure includes a compliant washer mounted about the shank and adjacent the head.

37. The system of claim 36, wherein each of the fastener element, the washer and the lock element is formed from a radiolucent material.

38. The system of claim 37, wherein the head of the fastener element is substantially disc-shaped, and the shank has substantially flat first and second substantially parallel surfaces, the surfaces defining ratchet teeth, the lock element.

39. The system of claim 37, wherein the washer has a coefficient of friction greater than a coefficient of friction of the fastener element.

40. The system of claim 37, wherein the washer defines a recessed area to receive a portion of the head of the fastener element.
